(19) United States
(12) Patent Application Publication
(10) Pub. No.: US 2023/0245787 A1
(43) Pub. Date: Aug. 3, 2023

(54) METHODS FOR PREDICTING SYNERGISTIC DRUG COMBINATION

(71) Applicant: GenomiCare Biotechnology (Shanghai) Co., Ltd, Shanghai (CN)

(72) Inventors: Qiang XU, Shanghai (CN); Guan WANG, Shanghai (CN); Yi ZHANG, Shanghai (CN); Bing WU, Shanghai (CN)

(21) Appl. No.: 18/159,923

(22) Filed: Jan. 26, 2023

Related U.S. Application Data
(63) Continuation of application No. PCT/CN2020/104900, filed on Jul. 27, 2020.

(51) Int. Cl. G16H 50/70; G16H 20/10; G16H 50/30; G16B 20/00

(52) U.S. Cl. CPC G16H 50/70; G16H 20/10; G16H 50/30; G16B 20/00

(57) ABSTRACT

A computer-implemented method of determining effects of drug combinations on treatment outcomes. A plurality of genomic and clinical variables are generated from the combination of comprehensive genomic data, EHR data, and clinical treatment data. Based on Cox Proportional Hazards model, independent risk factors, cumulative hazard-ratios, and p-values for the combination of a first drug and a second drug (or a biomarker) are calculated to determine the nature of the combination of the first drug and the second drug (or the biomarker) with respect to treating the disease.

### BACKGROUND

Drug combination has been widely used in treating diseases, including some of the most dreadful diseases such as cancer and AIDS. Oftentimes, a drug combination can have better therapeutic outcomes than single anticancer drug treatment. The rationale for drug combination includes improved therapeutic effect, dose and toxicity reduction, and the reduction or delay of drug resistance.

Due to the large number of available drugs, their complex (and often not well-understood) mechanism in treating diseases, and the complicated drug-drug interactions, finding a good combination of drugs having improved efficacy, toxicity and other properties of drug combinations can be extremely difficult. It is infeasible to experimentally screen possible drug combinations considering the limited resources.

There is a need to develop methods for improved reliability/accuracy in the prediction of properties of combination drugs.

### SUMMARY

The present disclosure provides computer-implemented methods to predict drug combinations using genomic data, treatment patterns, and clinical outcomes data.

In one aspect, the present disclosure provides a method of determining effects of drug combinations on treatment outcomes, which comprises: generating a plurality of genomic and clinical variables from the combination of (1) comprehensive genomic data, (2) EHR data, and (3) clinical treatment data, for each of a plurality of patients; wherein the plurality of patients comprise at least a first subset who have been treated with at least one first drug for a disease, and a second subset who have been treated with at least one second, different drug for the same disease, the first subset not entirely overlapping the second subset; setting up a plurality of two by two contingency tables in which rows are defined by the presence or absence of each of the plurality of genomic and clinical variables, and the columns are defined by the presence or absence of each of the first drug and the second drug; based on a Cox Proportional Hazards model, calculating independent risk factors, cumulative hazard-ratios, and p-values for the combination of the first drug and the second drug; and determining the nature of the combination of the first drug and the second drug as being one of additive, synergistic, and antagonistic with respect to treating the disease.

In another aspect, the present disclosure provides a method of determining drug effect on treatment outcome for a disease, comprising: generating a plurality of genomic and clinical variables from the combination of (1) comprehensive genomic data, (2) EHR data, and (3) clinical treatment data, for each of a plurality of patients; wherein some, but not all, of the plurality of patients share a common biomarker, and wherein some, but not all, of the plurality of patients have been treated with a same drug for a disease; based on the plurality of genomic and clinical variables and a two by two contingency table representing the following combinations: (1) the number of patients having the biomarker and having been treated with the drug, (2) the number of patients having the biomarker but having not been treated by the drug, (3) the number of patients not having the biomarker and having been treated with the drug, and (4) the number of patients not having the biomarker and not having been treated by the drug, using a Cox Proportional Hazards model to calculate independent risk factors, cumulative hazard-ratios, and p-values for the combination of the drug and the biomarker; and determining the nature of the combination of the drug and the biomarker as being one of additive, synergistic, and antagonistic with respect to treating the disease.

In further aspect, the present disclosure provides a method of determining effects of drug combinations on treatment outcomes, the method comprising: generating a plurality of genomic and clinical variables from the combination of (1) comprehensive genomic data, (2) EHR data, and (3) clinical treatment data, for each of a plurality of patients; wherein the plurality of patients comprise at least a first subset who have been treated with at least one first drug for a disease, a second subset who have been treated with at least one second, different drug for the same disease, and a third subset who have been treated with at least one third drug which are different from the first drug and different from the second drug for the same disease, each of the first, second, and third subsets not entirely overlapping with any of other subsets; setting up a plurality of two by two contingency tables in which rows are defined by the presence or absence of each of the plurality of genomic and clinical variables, and the columns are defined by the presence or absence of each of the first, second and third drug; based on a Cox Proportional Hazards model, calculating independent risk factors, cumulative hazard-ratios, and p-values for the combination of the first and the second drug, combination of the first and the second drug, and combination of the first and the second drug, and determining the nature of all possible binary combinations of the first, second and third drug as being one of additive, synergistic, and antagonistic with respect to treating the disease.

In some embodiments, whole-exome (WES) and transcriptome (RNA-Seq) sequencing of tumors of patients are first obtained. Bioinformatics analysis can be performed on the sequencing data to provide certain genomic features for each cancer patient, such as gene expression, loss of heterozygosity (LOH), copy number alteration (CNA), somatic and germline mutations, Microsatellite instability (MSI), tumor mutational burden (TMB), Chromosomal Variation, Mutational signatures, human Leukocyte Antigen Typing (HLA) and human pathogen. Demographics, tumor types/characteristics (biomarkers, stage, pathology), treatment (prescriptions, surgery, radiotherapy, diagnostic imaging, side effects/adverse events), and long-term survival outcome clinical variables can be obtained from real-world clinical electronic health records (EHRs).

Any of the steps or aspects of the methods disclosed herein can be carried out on a computer using one or more computer processors.

### BRIEF DESCRIPTION OF THE DRAWINGS

FIG. 1 is a flowchart illustrating data processing for comprehensive genomic bioinformatics analysis whole-exome sequencing (WES).

FIG. 2 is a flowchart illustrating data processing for transcriptome sequencing RNA-seq pipeline (RNA-seq).

FIG. 3 is a flowchart illustrating clinical high-throughput sequencing and bioinformatics analysis according to an embodiment of the present disclosure.

FIG. 4 is a flowchart illustrating real-world clinical electronic health records (EHRs) collection, clinical data entry and long-term follow-up according to an embodiment of the present disclosure.

FIG. 5 is a flowchart illustrating comprehensive genomic data being matched with real-world treatment patterns and clinical outcomes feature database and analysis workflow according to one embodiment of the present disclosure.

FIG. 6 is an example architecture of a computing device on which steps of the described methods of the present disclosure may be implemented or operated.

FIG. 7 is real-world survival data of treatment using PD-1/PD-L1 inhibitors in combination with Lenvatinib on certain Chinese HCC and ICC patients, and evaluation by methods described in the present disclosure.

FIG. 8 is real-world survival data of treatment using PD-1/PD-L1 inhibitors with genetic HLA-B*15:01 factor in certain Chinese Hepatocellular carcinoma, cholangiocarcinoma, Glioma, lung adenocarcinoma and soft tissue sarcoma patients, and evaluation by the methods described in the present disclosure.

### DETAILED DESCRIPTION OF CERTAIN EMBODIMENTS

Reference will now be made in detail to the present embodiments of the invention, examples of which are illustrated in the accompanying drawings.

In one aspect, the present disclosure provides a method of determining effects of drug combinations on treatment outcomes, which comprises: generating a plurality of genomic and clinical variables from the combination of (1) comprehensive genomic data, (2) EHR data, and (3) clinical treatment data, for each of a plurality of patients; wherein the plurality of patients comprise at least a first subset who have been treated with at least one first drug for a disease, and a second subset who have been treated with at least one second, different drug for the same disease, the first subset not entirely overlapping the second subset; setting up a plurality of two by two contingency tables in which rows are defined by the presence or absence of each of the plurality of genomic and clinical variables, and the columns are defined by the presence or absence of each of the first drug and the second drug; based on a Cox Proportional Hazards model, calculating independent risk factors, cumulative hazard-ratios, and p-values for the combination of the first drug and the second drug; and determining the nature of the combination of the first drug and the second drug as being one of additive, synergistic, and antagonistic with respect to treating the disease.

In another aspect, the present disclosure provides a method of determining effects of drug combinations on treatment outcomes, the method comprising: generating a plurality of genomic and clinical variables from the combination of (1) comprehensive genomic data, (2) EHR data, and (3) clinical treatment data, for each of a plurality of patients; wherein the plurality of patients comprise at least a first subset who have been treated with at least one first drug for a disease, a second subset who have been treated with at least one second, different drug for the same disease, and a third subset who have been treated with at least one third drug which are different from the first drug and different from the second drug for the same disease, each of the first, second, and third subsets not entirely overlapping with any of other subsets; setting up a plurality of two by two contingency tables in which rows are defined by the presence or absence of each of the plurality of genomic and clinical variables, and the columns are defined by the presence or absence of each of the first, second and third drug; based on a Cox Proportional Hazards model, calculating independent risk factors, cumulative hazard-ratios, and p-values for the combination of the first and the second drug, combination of the first and the second drug, and combination of the first and the second drug, and determining the nature of all possible binary combinations of the first, second and third drug as being one of additive, synergistic, and antagonistic with respect to treating the disease. A particular combination of two drugs can be selected for treating patients based on the determined nature of these possible binary combinations of drugs.

In another aspect, the present disclosure provides a method of determining drug effect on treatment outcome for a disease, comprising: generating a plurality of genomic and clinical variables from the combination of (1) comprehensive genomic data, (2) EHR data, and (3) clinical treatment data, for each of a plurality of patients; wherein some, but not all, of the plurality of patients share a common biomarker, and wherein some, but not all, of the plurality of patients have been treated with a same drug for a disease; based on the plurality of genomic and clinical variables and a two by two contingency table representing the following combinations: (1) the number of patients having the biomarker and having been treated with the drug, (2) the number of patients having the biomarker but having not been treated by the drug, (3) the number of patients not having the biomarker and having been treated with the drug, and (4) the number of patients not having the biomarker and not having been treated by the drug, using a Cox Proportional Hazards model to calculate independent risk factors, cumulative hazard-ratios, and p-values for the combination of the drug and the biomarker; and determining the nature of the combination of the drug and the biomarker as being one of additive, synergistic, and antagonistic with respect to treating the disease.

The disclosed method utilizes certain data sources, which can be provided by healthcare institutions, hospitals, clinics, medical practice groups, and patients. As an example, for evaluation efficacy of possible cancer drug combinations, data about cancer patients can be used. Tumor tissues can be collected from patients, pathology tests can be performed on the tissue, and the tissue can also be subject to genomic sequencing, such as whole-exome (WES) and transcriptome (RNA-Seq) sequencing. Bioinformatics analysis can be performed on the sequencing data to provide certain genomic features for each cancer patient, such as gene expression, loss of heterozygosity (LOH), copy number alteration (CNA), somatic and germline mutations, Microsatellite instability (MSI), tumor mutational burden (TMB), Chromosomal Variation, Mutational signatures, human Leukocyte Antigen Typing (HLA) and human pathogen.

Meanwhile, patient data from real-world clinical electronic health records (EHR) for the patients can be used to obtain demographics, medical history, medication and allergies, immunization status, laboratory test results, radiology images, vital signs, personal statistics like age and weight, etc. of the patients. Such patient data can be de-identified, processed, and stored into a database for use by clinical management software. Quality control and inspection may be performed on the patient data to reduce or eliminate errors.

Further, clinical treatment data can also be obtained about the patients. For example, for cancer patients, a patient may undergo one or more therapies and has been treated by one or more drugs for the cancer. The clinical treatment data can include prescriptions, surgery, radiotherapy, diagnostic imaging, side effects/adverse events, other treatment status and progress, as well as outcomes.

TABLE 1
Summary of Comprehensive Genomic Data

| Genomic | | Tools and |
|---|---|---|
| analysis | Description | databases |
| Somatic | The commercial Sentieon packages were | Sentieon |
| Mutation | applied to identify somatic mutations (SNP | hg19 VEP |
| | and Indel) in tumor compared with a | |
| | matched control blood sample from one | |
| | patient. Somatic mutations were annotated | |
| | by VEP hg19 version. | |
| Tumor Mutation | TMB was defined as the total number of | NA |
| Burden TMB | somatic nonsynonymous mutations in the | |
| | tumor exome. To calculate TMB | |
| | mutations/Mb, the total number of somatic | |
| | nonsynonymous mutations was normalized | |
| | to the total number of exonic megabases | |
| | sequenced. | |
| Microsatellite | MSI is a condition exhibited by certain | msisensor |
| instability MSI | tumors involving DNA mismatch repair | |
| | defects that lead to high mutation rates. | |
| Gene Copy | Normalized the number of reading counts | Inhouse |
| Number | for each genes' exonic region can reflect | development |
| Variation | copy numbers changes in tumor. Standard | |
| | normal distribution was used to normalized | |
| | five sources of bias that affect raw read | |
| | counts, including the size of exonic regions, | |
| | batch effect, sequencing data quantity and | |
| | quality, local GC content percentage and | |
| | genomic mappability. | |
| Chromosomal | Chromosomal Variations is defined as focal | Inhouse |
| variation | events and more complex arm level events. | development |
| Pathogen | Pathogenic microorganisms achieve their | Centrifuge |
| Taxomic | persistence in the human body by | |
| | integrating its genome into the human cell | |
| | genome leading to development of | |
| | carcinoma. | |
| Mutational | Mutation signatures are characteristic | COSMIC |
| Signature | combinations arising from a specific | signatures_v2 |
| | mutagenesis process such as DNA | database |
| | replication infidelity, exogenous and | |
| | endogenous genotoxins exposures, | |
| | defective DNA repair pathways and DNA | |
| | enzymatic editing. | |
| Loss of | Heterozygosity is the condition of having | Inhouse |
| heterozygosity | two different alleles at one locus. Loss of | development |
| (LOH) | heterozygosity is the phenomenon of | |
| | partially or completely losing one allele at | |
| | the locus caused by direct deletion, gene | |
| | conversion, or loss of chromosome. | |
| HLA Typing | HLA is a gene complex encoding human | Optitype |
| | major histocompatibility complex. It is | |
| | located on the short arm of chromosome 6 | |
| | (6p21.21). These cell surface proteins are | |
| | involved in the regulation of the human | |
| | immune system. | |
| Rare Germline | Pathogenic interpretation of Germline | Inhouse |
| Mutation | mutated genes was performed based on the | development |
| | ClinVar database. Cutoff mutation rate <1% | |
| | is a cutoff in The East Asian Population | |
| | from Exome Aggregation Consortium(ExAC), <2%. | |
| Homologous | Homologous recombination is a process of | scarHRD |
| Recombination | exchanging genetic materials between the | |
| Deficiency | two sister strands of DNA and was defined | |
| (HRD) | as the levels of homologous recombination | |
| | deficiency (telomeric allelic imbalance | |
| | HRD-TAI, loss off heterozygosity HRD-LOH, | |
| | number of large-scale transitions HRD-LST) | |
| | based on whole exome data. | |
| Gene expression | The determination of the pattern of genes | STAR, |
| | expressed at the level of genetic | StringTie2 |
| | transcription, and will take into account the | |
| | various expression measures produced: | |
| | count, FPKM (Fragments Per Kilobase Million), | |
| | TPM (Transcripts Per Million) | |
| Fusion | Fusion gene is a hybrid gene formed from | STAR-FUSION |
| | two independent genes through | |
| | chromosomal rearrangement. | |
| Immunopheno | The immune score is calculated by the TPM | immunophenogram |
| Score | of the immunogenic gene, reveals | |
| | genotype-immunophenotype relationships | |
| | and predictors of response to checkpoint | |
| | blockade | |
| Immune Cell | This method estimates the relative | ImmuCellAI |
| Infiltration | abundance of 24 immune cell types from | |
| Score | RNA data based on deviation of gene | |
| | signatures, with powerful and unique | |
| | function in tumor immune infiltration | |
| | estimation and immunotherapy response | |
| | prediction. | |
| Neoantigen | Neoantigens are antigens that are not | STAR-FUSION |
| | expressed in normal tissues, but expressed | |
| | only in tumor tissues, in this study, | |
| | neoantigens mainly include antigens | |
| | produced by mutant proteins. | |

TABLE 2
Clinical and follow-up variables from real-world
clinical electronic health records (EHRs).

| Clinical data | Description | Source |
|---|---|---|
| Survival data | Integration of the various sources of | EHRs |
| | real-world data (RWD), including EHRs, | |
| | clinical decision and support and | |
| | hospital-based systems, administrative | |
| | billing and claims databases, patient | |
| | registries, longitudinal studies, and | |
| | patient reported outcomes tools, will | |
| | yield a more robust dataset of real- | |
| | world evidence (RWE). | |
| Drug | Drugs for cancer and conditions related | EHRs |
| | to cancer. | |
| Demographics | Age, gender, and ethnicity | EHRs |
| data | | |
| Diagnosis data | biomarkers, stage and pathology | EHRs |
| Treatment data | prescriptions, surgery, radiotherapy, | EHRs |
| | diagnostic imaging, side effects/adverse | |
| | events | |
| Disease Main | The OncoTree tools is an open-source | OncoTree |
| Category | ontology that was developed at Memorial | |
| | Sloan Kettering Cancer Center (MSK) for | |
| | standardizing cancer type diagnosis from | |
| | a clinical perspective by assigning each | |
| | diagnosis a unique OncoTree code. | |
| Primary Site | A primary tumor is a tumor growing at | EHRs |
| | the anatomical site where tumor progression | |
| | began and proceeded to yield a cancerous | |
| | mass. | |
| Metastasis Site | Metastasis is a pathogenic agent's | EHRs |
| | spread from an initial or primary site | |
| | to a different or secondary site within | |
| | the host's body | |

Based on the comprehensive genomic data, EHR data, and real-world treatment data, a database can be built to match these data and generate a plurality of genomic and clinical variables.

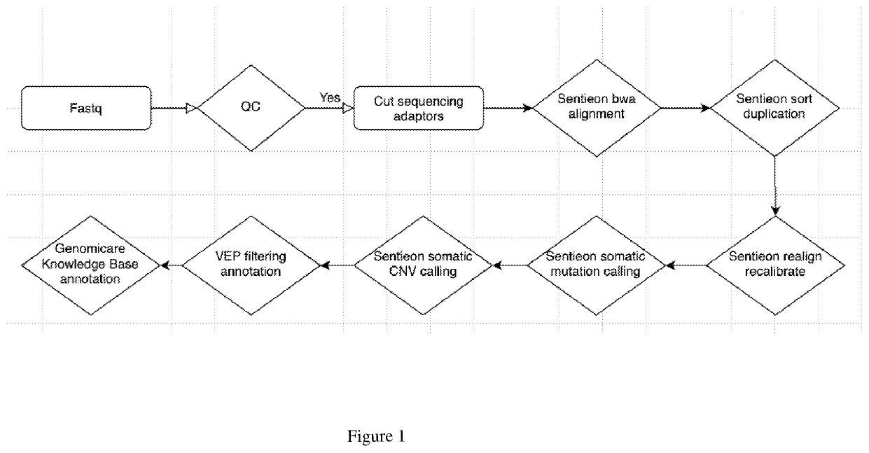

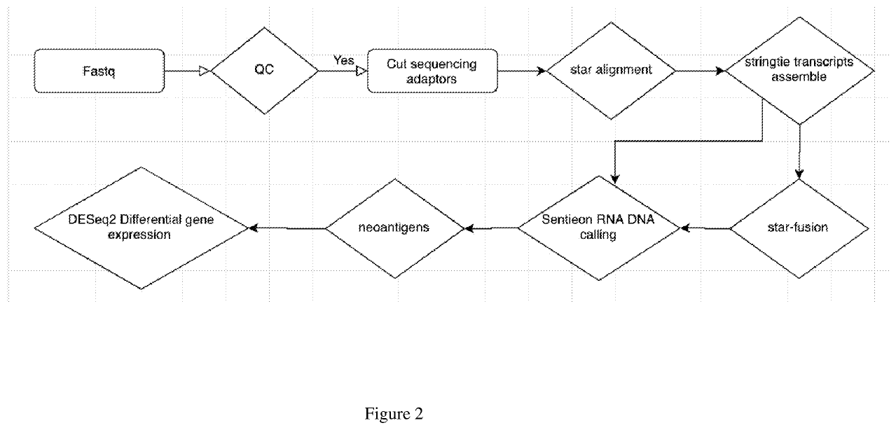

FIGS. 1 and 2 are flowcharts illustrating data processing for comprehensive genomic bioinformatics analysis whole-exome sequencing (WES) and transcriptome sequencing RNA-seq pipeline.

Then, a novel exhaustive Cox proportional hazards model (ECPH) model is used to evaluate all possible drug combinations with respect to their efficacy in prolonging patients' lives. In terms of efficacy, there can be three types of drug interaction: additive, synergistic, and antagonistic. Identifying the drug combination interactions in the clinical trial and/or real-world clinical data can help make the choice between sequential and simultaneous treatment and the design of new drug combinations.

Additive interaction means the effect of two chemicals is equal to the sum of the effect of the two chemicals taken separately. Synergistic interaction means that the effect of two substances/agents taken together is greater than the sum of their separate effect at the same doses. Antagonistic interaction means that the effect of two substances/agents is actually less than the sum of the effect of the two drugs taken independently of each other. By mathematic interaction definition, if the combination effect is greater than the mathematic probability of the two agents contributing independently (Synergistic), equal to the probability of their independent activities (Additive) or less than predicted (Antagonistic).

The Cox proportional-hazards (CPH) model is essentially a regression model used in medical research for investigating the association between the survival time of patients and one or more predictor variables. The CPH model extends survival analysis methods to assess simultaneously the effect of several risk factors on survival time.

The approach used in the present disclosure further extends the CPH model. In an example process, drugs used for fewer than one treatment cycle or the number of patients in any variables less than 15 were removed. Then, a 2×2 contingency table is set up, in which rows are defined by every unique genomic or clinical variable, and the columns are defined by drug variable (See Table 3 as an illustration). Then the CPH model (as described in “The Robust Inference for the Cox Proportional Hazards Model”, D. Y. Lin &L. J. Wei, Journal of the Am. Stat. Assoc., pp. 1074-1078, 1989) is used to calculate independent risk factors, cumulative hazard-ratios, and p-values for each drug combination table. Then these results are used predict and prioritize effective drug combinations with respect to additive, synergistic and antagonistic effects.

TABLE 3
2 × 2 contingency tables for all possible drug combinations pairs

| | Factor | Factor | |
|---|---|---|---|
| | B present | B Absent | |
| | (B+) | (B−) | Total |
| Drug A Treatment Group (A+) | 36 | 14 | 50 |
| Drug A free Treatment Group (A−)- | 30 | 25 | 55 |
| Total | 66 | 39 | 105 |

Additive, synergistic and antagonistic effect of factor/drug combinations in real-word clinical outcomes can be described as follows.

Additive combination definition: The HR score of the drug A+ plus factor B+ group is between the other two treatment groups (A+B− and A−B+). The P-value of the drug A+ plus factor B+ group is not statistically significant compared with the other two treatment groups (A+B− and A−B+).

Synergistic combination definition: The HR score of the drug A+ plus factor B+ group is smaller than the other two treatment groups (A+B− and A−B+). The P-value of the drug A+ plus factor B+ group has statistically significant compared with the other two treatment groups (A+B− and A−B+). The drug A+ plus factor B+ group is the statistically independent variable of the other two treatment groups (A+B− and A−B+).

Antagonistic combination definition: The HR score of the drug A+ plus factor B+ group is greater than the other two treatment groups (A+B− and A−B+). The P-value of the drug A+ plus factor B+ group has statistically significant compared with the other two treatment groups (A+B− and A−B+).

In the above definitions of different types of combinations, factor B can be a second drug which has been used to treat the patient cohort, or a certain characteristic of the patient cohort, for example, a genomic biomarker.

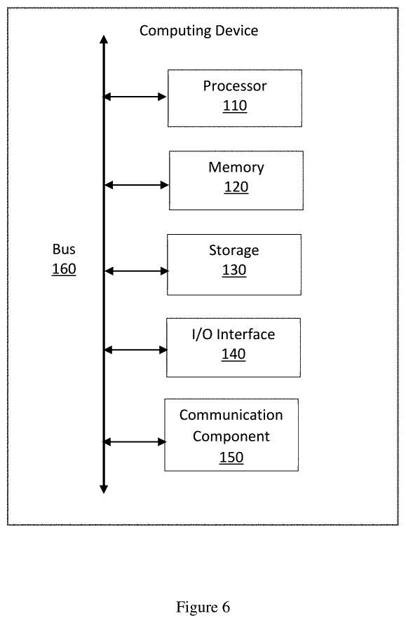

Any steps of the described methods can be performed on one or more computing devices (e.g., a workstation, a PC, a laptop, a mobile device, etc., or networked computers in a distributed environment, e.g., a cloud). As shown in FIG. 6, an example computing device 10 of the present disclosure includes a processor 110, memory 120, storage 130, an input/output (I/O) interface 140, a communication component 150, and a bus 160. Although this figure illustrates a particular computing device having a particular number of particular components in a particular arrangement, this disclosure contemplates any suitable computing device having any suitable number of any suitable components in any suitable arrangement. The processor can include hardware for executing instructions, such as those making up a computer program or application, for example, it may retrieve (or fetch) the instructions from an internal register, an internal cache, memory, storage; decode and execute them; and then write one or more results to internal register, internal cache, memory, or storage. In particular embodiments, software executed by processor 110 may include an operating system (OS) (e.g., Windows, Unix, MacOS, etc.), and applications designed for implement the methods herein. In some embodiments, the memory 120 can include main memory for storing instructions for the processor to execute or data for processor to operate on. One or more buses 160 may connect the processor with the memory. The memory 120 can include random-access memory (RAM). This RAM may be volatile memory, where appropriate. Where appropriate, this RAM may be dynamic RAM (DRAM) or static RAM (SRAM). The storage 130 can include non-volatile and/or non-transient mass storage or media for data or instructions, for example HDD, SSD, flash memory, optical medium, etc., or a combination of two or more thereof. The I/O interface 140 can include hardware, software, or both providing one or more interfaces for communication between two or more computing devices and one or more I/O devices. The communication component 150 can include hardware, software, or both providing one or more interfaces for communication (such as, for example, packet-based communication) between the computing device with another computing device, for example, a network interface controller (NIC) or network adapter for communicating with an Ethernet or other wire-based network or a wireless NIC (WNIC), wireless adapter for communicating with a wireless network, such as a Wi-Fi network or cellular network, or a combination of two or more thereof. The bus 160 can include hardware, software, or both coupling components of the personal computing device to each other, for example, a graphics bus, an Enhanced Industry Standard Architecture (EISA) bus, a front-side bus (FSB), or another suitable combinations.

### EXAMPLES

The described methods are validated by the following examples.

1. Synergistic Combination of Lenvatinib with PD-1/PD-L1 Immune Checkpoint Inhibitors for Hepatocellular Carcinoma (HCC) and Intrahepatic Cholangiocarcinoma (ICC).

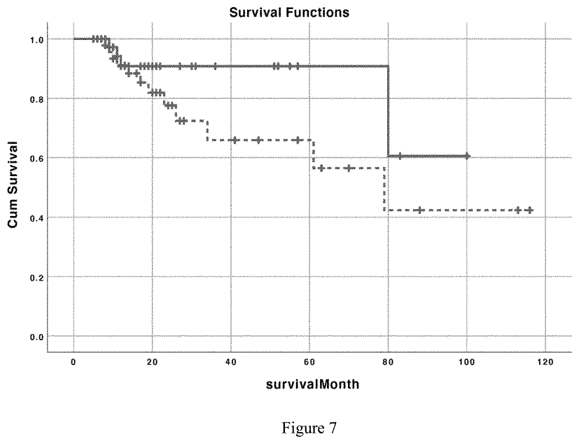

FIG. 7 shows real-world survival data of PD-1/PD-L1 inhibitors in combination with Lenvatinib in 105 Chinese HCC and ICC patients, and evaluation by the ECPH model-based method described in the present disclosure. The top survival curve is the 46 Lenvatinib plus PD-1/PD-L1 inhibitors patient group versus the bottom survival curve for 59 patient group which were treated with only PD-1/PD-L1 inhibitors. Lenvatinib is a statistically independent good prognostic factor in HCC and ICC after PD-1/PD-L1 inhibitors treatment. The combination patient group (Lenvatinib+PD-1/PD-L1) showed a statistically significant better in survival than either treatment with Lenvatinib or PD-1/PD-L1 group (PD-1/PD-L1+ and Lenvatinib+ group: HR: 0.278, P-value: 0.008; PD-1/PD-L1+ and Lenvatinib− group: HR: 0.503, P-value: 0.117; PD-1/PD-L1− and Lenvatinib+ group: HR: 1.00 P-value: 0.977).

In this example, data were collected and analyzed in the following steps:

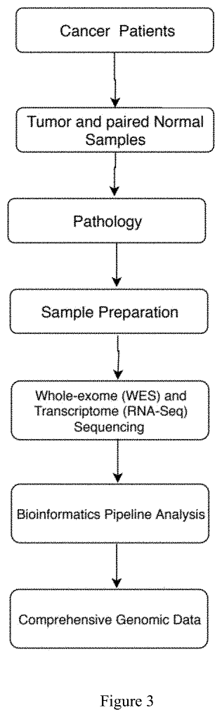

(1) Clinical high-throughput sequencing and bioinformatics analysis according to the flow chart shown in FIG. 3.

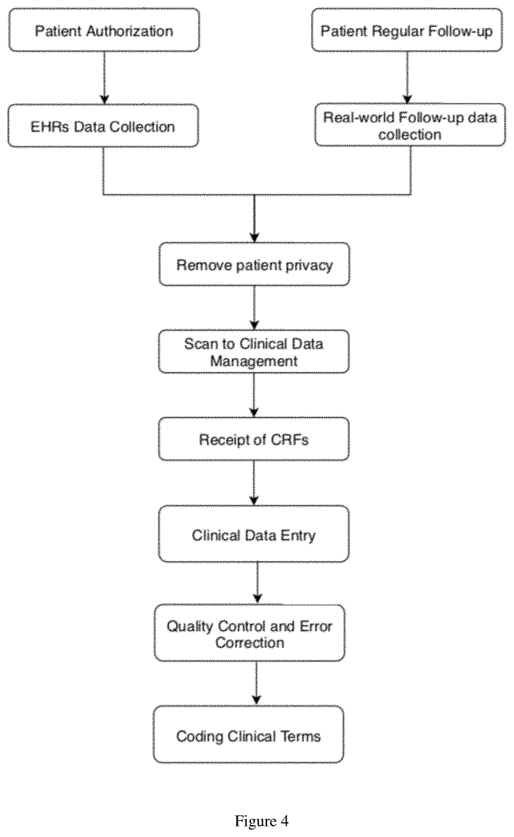

(2) Real-world clinical electronic health records (EHRs) collection, clinical data entry and l long-term follow-up, according to the flowchart shown in FIG. 4.

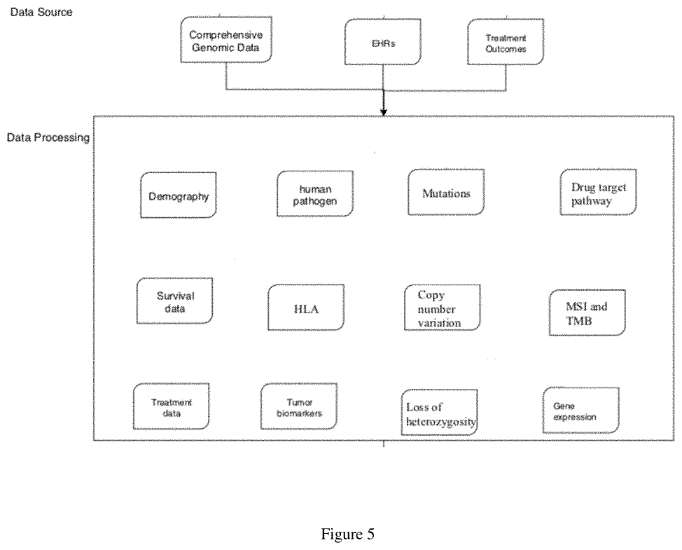

(3) Comprehensive genomic data is matched with real-world treatment patterns and clinical outcomes feature database and analysis workflow, as shown in FIG. 5.

(4) A large one-hot encoding matrix(˜10,000*10,000) of comprehensive genomic and clinical factors. Based on the one-hot encoding matrix, the combined effects of all factors such as: age, gender, gene mutation, and drug treatment can be obtained.

A sample snippet of one-hot matrix encoding in shown in the below Table.

TABLE 4
Sample encoding

| ID | R1 | R2 | S1 | S2 | C1 | M1 | T1 | D1 | D2 | D3 |
|---|---|---|---|---|---|---|---|---|---|---|
| A04262 | 1 | 13 | 1 | 0 | 0 | 0 | 0 | 1 | 0 | 1 |
| A00964 | 0 | 28 | 0 | 1 | 0 | 0 | 0 | 1 | 0 | 0 |
| A04736 | 0 | 16 | 0 | 1 | 0 | 0 | 0 | 1 | 0 | 0 |
| A04764 | 0 | 15 | 0 | 1 | 0 | 1 | 0 | 0 | 0 | 0 |
| A04859 | 0 | 15 | 0 | 1 | 0 | 0 | 1 | 0 | 0 | 0 |
| A04884 | 0 | 15 | 0 | 1 | 0 | 0 | 0 | 1 | 0 | 0 |

Wherein in the above table, the column headers represent the below variables:
R1: death_observed
R2: survival Month
S1: gender_Female
S2: gender_Male
C1: stage_1
M1: amp_ERBB2
T1: chemotherapy
D1: Apatinib
D2: PD-1
D3: Lenvatinib

(5) Applying exhaustive Cox proportional hazards model (ECPH) model

a. Patients are divided into four categories based on the combined use of the two drugs. For an example: Patients treated with Lenvatinib and without Sorafenib were defined as the Lenvatinib treatment group. Patients treated with Sorafenib and without Lenvatinib were defined as the Sorafenib treatment group. Patients treated with Sorafenib and Lenvatinib were defined as the Sorafenib-Lenvatinib treatment group. Patients treated without Sorafenib and Lenvatinib were defined as Sorafenib-Lenvatinib free treatment group.

b. 2×2 contingency tables were calculated for all possible drug combinations pairs: Millions of possible combinations of Lenvatinib.

c. Cox PH model (The Robust Inference for the Cox Proportional Hazards Model D. Y. Lin &L. J. Wei Pages 1074-1078) was used to calculate independent risk factors, cumulative hazard-ratios, and p-values for each drug combination table. Briefly, the hazard function can be interpreted as the risk of dying at time t. It can be estimated as follow: h(t)=h₀(t)×exp(b₁x₁+b₂x₂+ . . . +bₚxₚ) where,
t represents the survival time.
h(t) is the hazard function determined by a set of p covariates (x₁, x₂, . . . , xₚ)
the coefficients (b₁, b₂, . . . , bₚ) measure the impact (i.e., the effect size) of covariates.
the term h₀ is referred to the baseline hazard. It corresponds to the value of the hazard if all the xᵢ are equal to zero (the quantity exp(0) equals 1). The ‘t’ in h(t) reminds us that the hazard may vary over time.

The Cox PH model can be written as a multiple linear regression of the logarithm of the hazard on the variables xᵢ, with the baseline hazard being an ‘intercept’ term that varies with time. The average hazard rate of the interval was used in which the number of patients dying per unit time in the interval is divided by the average number of survivors at the midpoint of the interval:

h(t)=number of patients dying per unit time in the interval/((number of patients surviving at t)−(number of deaths in the interval)/2)

The hazard ratio of the patient receiving the experimental drug and the one receiving placebo is:

h(t|x₁=1)/h(t|x₁=0)=exp(bᵢ)

The hazard ratios (HR) are defined as the quantities exp(bᵢ). Thus, the two treatments are equally effective if HR=1 and the experimental drug introduces lower (higher) risk for survival than placebo if HR<1 (HR>1). The function coxph (R survival package) can be used to compute the Cox proportional hazards regression model in R. (https://cran.r-project.org/web/packages/survival/survival.pdf). Using Contingency Table A below for an example, three treatment groups HR scores were obtained: Sorafenib+ and Lenvatinib+ group: HR: 1.35, P-value: 0.334; Sorafenib+ and Lenvatinib− group: HR: 0.76, P-value: 0.388; Sorafenib− and Lenvatinib+ group: HR: 0.56 P-value: 0.058. These three results were used to predict and prioritize effective drug combinations with respect to additive, synergistic and antagonistic effects (according to the above additive, synergistic and antagonistic combination definition), as well as explore the dynamics of combination therapy and its role in combating drug resistance in cancer treatments.

The below three tables are shown as calculation examples:

Contingency Table A

| | | Sorafenib | |
|---|---|---|---|
| | Sorafenib | Free | |
| | Treatment | Treatment | Total |
| Lenvatinib Treatment | 29 | 89 | 118 |
| Lenvatinib free Treatment | 63 | 491 | 554 |
| Total | 92 | 580 | |

Sorafenib+ and Lenvatinib+ group: HR: 1.35, P-value: 0.334; Sorafenib+ and Lenvatinib− group: HR: 0.76, P-value: 0.388; Sorafenib− and Lenvatinib+ group: HR: 0.56 P-value: 0.058

Contingency Table B

| | | Regorafenib | |
|---|---|---|---|
| | Regorafenib | Free | |
| | Treatment | Treatment | Total |
| Lenvatinib Treatment | 9 | 109 | 118 |
| Lenvatinib free Treatment | 24 | 543 | 567 |
| Total | 33 | 652 | |

Regorafenib+ and Lenvatinib+ group: HR: 1.88, P-value: 0.475; Regorafenib+ and Lenvatinib− group: HR: 0.99, P-value: 0.984; Regorafenib− and Lenvatinib+ group: HR: 0.74 P-value: 0.226

Contingency Table C

| | | PD-1/PD-L1 | |
|---|---|---|---|
| | PD-1/PD-L1 | Free | |
| | Treatment | Treatment | Total |
| Lenvatinib Treatment | 46 | 96 | 142 |
| Lenvatinib free Treatment | 59 | 625 | 684 |
| Total | 105 | 721 | |

PD-1/PD-L1+ and Lenvatinib+ group: HR: 0.278, P-value: 0.008; PD-1/PD-L1+ and Lenvatinib− group: HR: 0.503, P-value: 0.117; PD-1/PD-L1− and Lenvatinib+ group: HR: 1.00 P-value: 0.977

Synergistic Combination Definition Used in this Example:

The HR score of the PD-1/PD-L1+ and Lenvatinib+ group is smaller than the other two treatment groups (HR: 0.278<PD-1/PD-L1+ and Lenvatinib− group: HR: 0.503 and PD-1/PD-L1− and Lenvatinib+ group: HR: 1.00). The P-value of the PD-1/PD-L1+ and Lenvatinib+ group has statistically significant compared with the other two treatment groups (P-value: 0.008<PD-1/PD-L1+ and Lenvatinib− group: P-value: 0.117 and PD-1/PD-L1− and Lenvatinib+ group P-value: 0.977). The PD-1/PD-L1 and Lenvatinib group is the statistically independent variable (independence of Chi-square test: P-value: 0.223). These results show the treatment with Lenvatinib plus anti-PD-1/PD-L1 treatment induced significant antitumor activity compared with Lenvatinib or anti-PD-1 treatment alone. Our ECPH model provide a real-world scientific rationale for combination therapy of Lenvatinib with anti-PD-1/PD-L1 blockade to improve cancer immunotherapy.

Such validation can also find support in the following references:
Ref 1. Lenvatinib plus anti-PD-1 antibody combination treatment activates CD8+ T cells through reduction of tumor-associated macrophage and activation of the interferon pathway. 2.776 PLoS One. 2019 Feb. 27; 14(2):e0212513. doi: 10.1371/journal.pone.0212513. eCollection 2019. The authors here show that lenvatinib modulates cancer immunity in the tumor microenvironment by reducing TAMs and, when combined with PD-1 blockade, shows enhanced antitumor activity via the IFN signaling pathway.
Ref 2. Phase II study of lenvatinib plus pembrolizumab for disease progression after PD-1/PD-L1 immune checkpoint inhibitor in metastatic clear cell renal cell carcinoma (mccRCC): Results of an interim analysis. ESMO 2019 Congress Annals of Oncology (2019) 30 (suppl_5): v475-v532. 10.1093/annonc/mdz253. At data cutoff (Mar. 29, 2019), the first 33 pts enrolled were followed for ≥12 weeks for response evaluation, and 24 (73%) pts were still on study treatment. The objective response rate was 52%, the disease control rate was 94%, and most pts had tumor shrinkage. Median follow-up time for progression-free survival was 4.2 months.
Ref 3. Lenvatinib plus pembrolizumab in patients with advanced endometrial cancer: an interim analysis of a multicentre, open-label, single-arm, phase 2 trial. Lancet Oncol. 2019 May; 20(5):711-718. doi: 10.1016/S1470-2045(19)30020-8. Epub 2019 Mar. 25. Here, Lenvatinib plus pembrolizumab showed anti-tumour activity in patients with advanced recurrent endometrial cancer with a safety profile that was similar to those previously reported for lenvatinib and pembrolizumab monotherapies, apart from an increased frequency of hypothyroidism. Lenvatinib plus pembrolizumab could represent a new potential treatment option for this patient population, and is being investigated in a randomized phase 3 study.

2. Antagonistic Combination (Drug Resistance) Genetics HLA Biomarker with PD-1/PD-L1 Immune Checkpoint Inhibitors.

In this example, a specific HLA-B biomarker is considered a second factor and its combination with the treatment of a PD-1/PD-L1 drug is evaluated in a similar manner as outlined above. For example, a 2 by 2 contingency table can be set up as follows:

| | | PD-1/PD-L1 | |
|---|---|---|---|
| | PD-1/PD-L1 | Free | |
| | Treatment | Treatment | Total |
| HLA-B*15:01 biomarker present | 47 | 406 | 453 |
| HLA-B*15:01 biomarker free | 398 | 430 | 828 |
| Total | 445 | 836 | |

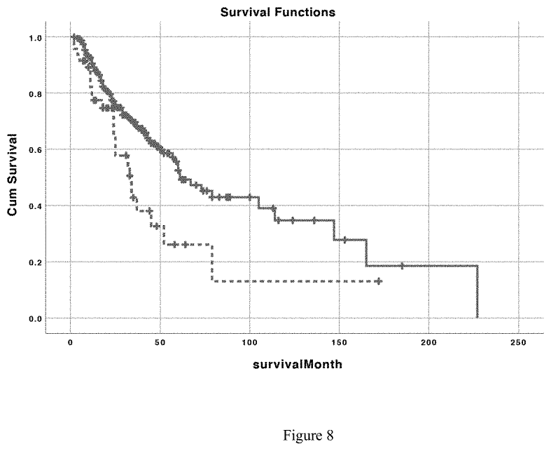

FIG. 8 shows real-world survival data of PD-1/PD-L1 inhibitors with genetic HLA-B*15:01 factor in 445 Chinese Hepatocellular carcinoma, cholangiocarcinoma, Glioma, lung adenocarcinoma and soft tissue sarcoma patients, and evaluation by the ECPH model-based methods described in the present disclosure. The bottom survival curve is the 47 HLA-B*15:01 plus PD-1/PD-L1 inhibitors patient group versus the top curve 398 patient group without HLA-B*15:01 treated with PD-1/PD-L1 inhibitors. HLA-B*15:01 is a statistically independent poor prognostic factor in main solid tumor after PD-1/PD-L1 inhibitors treatment. The combination treatment patient group (HLA-B*15:01+PD-1/PD-L1) showed a statistically significant worse in survival than either HLA-B*15:01 positive or treatment with PD-1/PD-L1 group (PD-1/PD-L1+ and HLA-B*15:01+ group: HR: 1.880 P-value: 0.007; PD-1/PD-L1+ and HLA-B*15:01-group: HR: 0.58 P-value: 0.069; PD-1/PD-L1− and HLA-B*15:01+ group: HR: 1.15 P-value: 0.442).

Such validation can also find support in the following reference: Patient HLA class I genotype influences cancer response to checkpoint blockade immunotherapy. (Science. 2018 Feb. 2; 359(6375):582-587. doi: 10.1126/science.aao4572. Epub 2017 Dec. 7.) In this paper, it is observed that two independent melanoma cohorts, patients with the HLA-B44 supertype had extended survival, whereas the HLA-B62 supertype (including HLA-B*15:01) or somatic loss of heterozygosity at HLA-I was associated with poor outcome.

It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

What is claimed is:

1. A computer-implemented method of determining effects of drug combinations on treatment outcomes, comprising:
generating a plurality of genomic and clinical variables from the combination of (1) comprehensive genomic data, (2) EHR data, and (3) clinical treatment data, for each of a plurality of patients; wherein the plurality of patients comprise at least a first subset who have been treated with at least one first drug for a disease, and a second subset who have been treated with at least one second, different drug for the same disease, the first subset not entirely overlapping the second subset;
based on the plurality of genomic and clinical variables and a two by two contingency table representing the number of patients falling in the four different combinations of treatments comprising (1) the number of patients having been treated both by the first drug and second drug, (2) the number of patients having been treated by the first drug but not the second drug, (3) the number of patients having been treated by the second drug but not the first drug, and (4) the number of patients having not been treated by either the first drug or the second drug, using a Cox Proportional Hazards model to calculate independent risk factors, cumulative hazard-ratios, and p-values for the combination of the first drug and the second drug; and
determining the nature of the combination of the first drug and the second drug as being one of additive, synergistic, and antagonistic with respect to treating the disease.

2. The method of claim 1, further comprising clinically testing the combination of the first drug and the second drug in treating the disease on a group of subjects if the combination of the first drug and the second drug has been determined to be synergistic.

3. The method of claim 1, wherein the genomic and clinical variables comprise one of: gene expression, loss of heterozygosity (LOH), copy number alteration (CNA), somatic and germline mutations, Microsatellite instability (MSI), tumor mutational burden (TMB), Chromosomal Variation, Mutational signatures, human Leukocyte Antigen Typing (HLA), and human pathogen.

4. A computer-implemented method of determining drug effect on treatment outcomes, comprising:
generating a plurality of genomic and clinical variables from the combination of (1) comprehensive genomic data, (2) EHR data, and (3) clinical treatment data, for each of a plurality of patients; wherein some, but not all, of the plurality of patients share a common biomarker, and wherein some, but not all, of the plurality of patients have been treated with a same drug for a disease;
based on the plurality of genomic and clinical variables and a two by two contingency table representing the following combinations: (1) the number of patients having the biomarker and having been treated with the drug, (2) the number of patients having the biomarker but having not been treated by the drug, (3) the number of patients not having the biomarker and having been treated with the drug, and (4) the number of patients not having the biomarker and not having been treated by the drug, using a Cox Proportional Hazards model to calculate independent risk factors, cumulative hazard-ratios, and p-values for the combination of the drug and the biomarker; and
determining the nature of the combination of the drug and the biomarker as being one of additive, synergistic, and antagonistic with respect to treating the disease.

5. A computer-implemented method of determining effects of drug combinations on treatment outcomes, comprising:
generating a plurality of genomic and clinical variables from the combination of (1) comprehensive genomic data, (2) EHR data, and (3) clinical treatment data, for each of a plurality of patients; wherein the plurality of patients comprise at least a first subset who have been treated with at least one first drug for a disease, a second subset who have been treated with at least one second, different drug for the same disease, and a third subset who have been treated with at least one third drug which is different from the first drug and different from the second drug for the same disease, each of the first, second, and third subsets not entirely overlapping with any of other subsets;
based on the plurality of genomic and clinical variables and contingency tables including information of patients having been treated by one or more of the first, second and third drugs, and using a Cox Proportional Hazards model, calculating independent risk factors, cumulative hazard-ratios, and p-values for the combination of the first and the second drug, the combination of the first and the second drug, and the combination of the first and the second drug, and
determining the nature of all possible binary combinations of the first, second and third drug as being one of additive, synergistic, and antagonistic with respect to treating the disease.

6. The method of claim 5, further comprising:
selecting a combination of two drugs based on the determined nature of all possible binary combinations of drugs.
